(19) United States
(12) Patent Application Publication
(10) Pub. No.: US 2003/0187343 A1
(43) Pub. Date: Oct. 2, 2003

(54) Force feedback tonometer

(76) Inventors: Oscar Cuzzani, Calgary (CA); Andrew J. Barker, Calgary (CA); Donald E. James, Calgary (CA)

Correspondence Address: SEAN W. GOODWIN, 237- 8TH AVE. S.E., SUITE 360, THE BURNS BUILDING, CALGARY, AB, T2G 5C3, CA

(21) Appl. No.: 10/400,869

(22) Filed: Mar. 28, 2003

Related U.S. Application Data
(60) Provisional application No. 60/367,767, filed on Mar. 28, 2002.

(51) Int. Cl. A61B 3/16

(52) U.S. Cl. 600/399

(57) ABSTRACT

Apparatus and method for measuring intraocular pressure (IOP). comprising a vibrator which transmits a vibrational energy into an eyeball through the eyelid and measures at least one of a force or phase response in the eyeball. The measurements are taken by placing the tonometer against the eyelid to induce vibration in the underlying eyeball. A force transducer coupled to the vibrator measures the response of the eyeball from which a vibrational impedance of the eye is determined. Intraocular pressure is then calculated based on the vibrational impedance. In a preferred use of the apparatus, the tonometer is calibrated against a known intraocular pressure measurement permitting the patient to take subsequent relative IOP measurements at home or otherwise outside a traditional medical setting without the need for anesthetic or fear of infection.

### FIELD OF THE INVENTION

The present invention relates to apparatus and method for the acquisition of physical, physiological and structural characteristics of an eyeball and more particularly for determining a measure of the intraocular pressure of the eye. More particularly, vibration is induced in the eye and a force transducer is applied to establish measures indicative of IOP.

### BACKGROUND OF THE INVENTION

Measuring the intraocular pressure (IOP) of an eye is a measurement of the pressure of the fluid inside the eye cavity. It is advantageous to monitor IOP as it is an indicator of the health of the eye. Excessively high IOP can be associated with optic nerve damage, such as in the case of glaucoma.

An eyeball may be deemed analogous to an elastic vessel filled with a fluid of a substantially incompressible nature. One can compare such an elastic vessel to a balloon having extensible walls where any increase in volume in the fluid will produce a change in the internal pressure that in turn will expand the vessel wall. Fluids inside the eye circulate in a substantially continuous fashion and an increase in the influx of fluids normally accompanies a similar increase in the outflow of fluid. In cases where the outflow does not keep up with inflow, an increase in internal pressure and an expansion of the vessel or eye will occur. In situations where the rigidity of the vessel wall is increased, two effects are observed: increases in the internal pressure are greater per increase in fluid inflow; and the overall expansion of the volume of the eye is smaller.

The change in the expansion of the eye depends on the extensibility of the vessel walls. The more extensible the wall, the greater the increase in eye volume. The less extensive the wall, the more the fluid pressure increases.

Most often in biomedicine, IOP is not be measured directly, because of the invasive nature of placing a pressure sensor in the fluid of the eyeball. Therefore, determination of pressure is typically attempted using less invasive, alternative methods. Consequently, measuring intraocular pressure directly, continuously, and non-invasively is important, but is difficult to achieve.

Moderately invasive measurements are known and have already been conducted. Contacting tonometers have been used extensively by the medical community for many years. Their attractiveness however is offset by the need to have direct mechanical contact with the eye, thus requiring an anesthetic. The requirement for contact and the resulting deformation of the eye introduces errors in the determination of IOP due to tear formation, change in eye volume due to compression, and as a result, the variance of the physical properties of the cornea. Such prior art is described in U.S. Pat. Nos.; 2,519,681; 3,049,001; 3,070,087; 3,192,765.

Various other attempts have been made to measure IOP discreetly or continuously by means of more indirect methods. Indirect methods have the advantage of being non-invasive, or at least less invasive than indentation and applanation tonometry. One such method introduces a sharp pulse of air onto the eye, while measuring the resulting deformation (U.S. Pat. No. 3,181,351). Such indirect methodology usually suffers from two limitations: lack of accuracy and/or lack of absolute value in the measurement.

Typically, patients having eye diseases such as glaucoma which affect the IOP may require frequent monitoring of IOP. Thus, what is needed is a noninvasive method for measuring the IOP that can safely be performed by the patient or others outside the usual medical setting, such as in the patient's home.

### SUMMARY OF THE INVENTION

Intraocular pressure (IOP) is determined through the eyelid using unique apparatus for transmission of mechanical energy, preferably vibration, to the eyeball. A measurement of vibrational responses induced in the eyeball are used to calculate vibrational impedance of the eyeball, which is a function of the IOP.

The advantages of this technique include simplicity and safety which permit a patient to monitor IOP outside of a conventional clinical environment and most particularly, at home.

In accordance with an embodiment of the present invention, a tonometer is provided for the measurement of IOP which uses a vibrator, such as a solenoid having a constant output amplitude and being driven by an oscillator, and controlled by a microprocessor or computer such that the output amplitude, the frequency and phase is known. The vibrator is connected or coupled to a force sensor, such as a force transducer or strain gauge, which is used to measure the feedback such as vibrational responses of the eye. More particularly, the force sensor measures at least one of a force response or a phase response.

In a broad aspect of the invention, a method is provided for determining measurement representing the IOP of an eye comprising the steps of: contacting an eyelid with a mechanical energy transmission means such as a vibrator capable of producing a constant amplitude and a range of frequencies for inducing vibration in at least a portion of an underlying eyeball; providing means for measuring a dimensional vibration response in the eyeball for establishing measures indicative of vibrational impedance; and calculating the intraocular pressure as a function of the vibrational impedance.

Preferably, the energy transmission means is a vibrator coupled to a force transducer for measurement of the vibrational response of the eye. More preferably, the force transducer measures at least one of a force or a phase response of the eye for establishing vibration impedance as a characteristic indicative of intraocular pressure. A static force sensor can also be used to ensure adequate force is used in applying the vibrator to the eyelid, thus ensuring adequate vibration is induced in the eyeball and a vibrational response is detected.

The method is understood to be accomplished with a variety of apparatus which is known to those skilled in the art. Namely, in a broad aspect of the invention, a force feedback tonometer is provided comprising: a mechanical energy transmission means such as a solenoid capable of producing a constant amplitude, variable frequency output for inducing vibration in at least a portion of an eyeball when positioned against an eyelid overlying the eyeball; a device for measuring a dimensional vibration response in the eyeball for establishing measures indicative of vibrational impedance; and means for calculating the intraocular pressure as a function of the measures indicative of vibrational impedance. Preferably, the energy transmission means is a vibrator coupled to a force transducer for measurement of the dimensional vibration response of the eye.

In use, a vibrating shaft or protuberance of the tonometer is placed gently in contact with the eyelid. Vibration is thus passed through the eyelid to the underlying eyeball, over a range of frequencies of interest, and the vibrational response of the eye is measure by the force transducer, which is mechanically coupled thereto. The vibrational impedance of the eyeball is determined by a microprocessor or computer using the applied vibrational characteristics and the measured responses. A definite association exists between the vibrational impedance and the IOP.

Optionally, for further normalizing the vibrational response, and contiguous with the vibrational impedance measurement, a laser interferometer is used to measure the geometry of the eye including an axial length of the eye from which the volume of the eye is deduced. Also the cornea thickness can be measured, from which additional mechanical properties such as the elasticity are deduced.

These measurements are more accurate than are possible by merely measuring the changes occurring in the corneal curvature or the force or time required to indent or flatten it. The reason for this is that when acoustic energy is used, it does not change the volume of the eye and thus does not substantially affect the pressure.

The IOP is measured by measuring vibrational properties of the cornea or eye as a whole. Characteristics which are identifiable and responsive to changes in IOP can be used for normalizing the IOP by removing the effect of each eye's own physical characteristics include: the physical three-dimensional response to the exciting vibration, the phase lag of the response with respect to the exciting force and the amplitude and/or shape of the phase response.

In applying the properties determined above, the method further comprises the step of determining the vibration response of the vibrating eye as a function of the axial length of the eye which can be related to the eye's volume, and the mechanical properties of the eye. Additionally, an elastic modulus of the vibrating eye is determined as a function of the thickness of the cornea and the water content of the cornea. Accordingly, most preferably, the IOP is determined as a function of the vibrational response, the mechanical properties and the geometry.

More preferably, the method further comprising the steps of: providing a laser interferometer for producing a measuring beam and interference patterns from a plurality of beams reflected back to the interferometer; and determining the path length between at least two of the reflected beams for establishing an axial length of the eye as a geometric characteristic of the eye. One can apply the axial length of the eye for establishing characteristics indicative of at least the volume of the eye. More particularly, the method comprises determining path lengths between at least two of the reflected beams for establishing a corneal thickness as a geometric characteristic of the eye.

### BRIEF DESCRIPTION OF THE DRAWINGS

FIG. 1 is a block diagram of a vibrational transducer exciting an eye, at constant amplitude, while a force transducer measures the magnitude and phase of the force;

FIGS. 2a and 2b illustrate an amplitude and phase of a force applied to a pig's eye, driven at constant amplitude, and under two different induced IOPs, more particularly

FIG. 2a is illustrative of a pig's eye having a low intraocular pressure; and

FIG. 2b is illustrative of a pig's eye having a high intraocular pressure; and

FIG. 3 is a block diagram of an optional laser interferometer measuring both the axial length and the cornea thickness of the eye.

### DETAILED DESCRIPTION OF THE PREFERRED EMBODIMENT

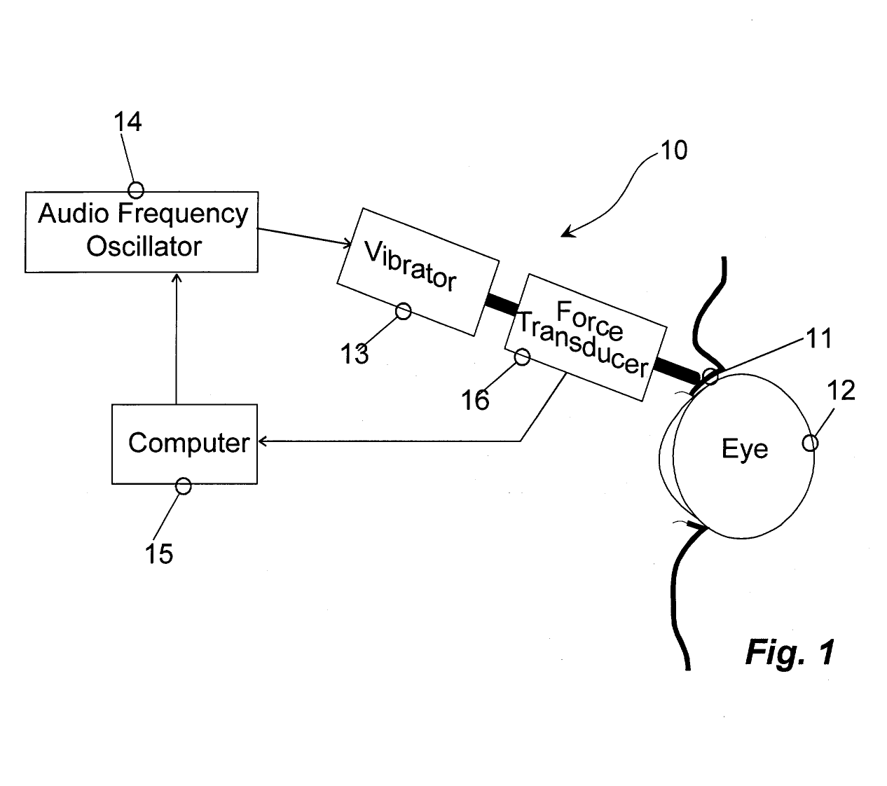

As shown in FIG. 1 and in accordance with the present invention, a tonometer 10 for measurement of intraocular pressure (IOP) is provided which can be applied to an eyelid 11 and does not require direct contact with an eyeball 12.

Mechanical energy, in this case a vibrational force, is transmitted to the eyeball 12 through the eyelid 11. The response of the eyeball 12 to the mechanical energy is related to the characteristics of the eyeball 12 and particularly to the IOP. When the vibrational force is applied to excite the eyeball, the resulting oscillation or vibrational response in the eyeball is measured. The vibrational force applied to the eyeball 12 is swept through a range of frequencies. The vibrational response is detected as a force feedback. At differing IOP, the frequency at which the force reaches a minimum shifts. Further, an inflection point occurs in a phase curve, and a phase peak, are also shifted in relation to the IOP.

Having reference again to FIG. 1, the tonometer 10, according to a preferred embodiment of the invention is shown. A vibrator 13 is driven by an audio frequency oscillator 14. The oscillator 14 is controlled by a microprocessor or computer 15 to produce a constant amplitude output over a range of frequencies of interest. Simultaneously, the computer 15 receives measures of the vibrational response from a mechanically coupled force transducer 16 for dynamically determining a vibrational impedance of the eye which is used to calculate measures indicative of the intraocular pressure. Preferably, the force transducer 16 measures at least one of a force and a phase response in the eyeball 12. The phase of the vibrator and the phase of the sensed force can be compared.

In use, the vibrational energy is transferred to the eyeball 12 by gently pressing a shaft 17 extending from the vibrator 13 against the eyelid 11. The frequency of the vibration determined by the oscillator 14 is swept across the range of frequencies of interest as the shaft 17 maintains contact with the eyelid 11. The response of the eyelid is not a substantial factor in determining the response of the eyeball 12 beneath.

More preferably, a static force sensor, whether the same dynamic force sensor 16 or discreet sensor (not shown), is used to ensure adequate force is used to apply the vibrator to the eyelid 11, thus ensuring adequate vibration induced in the eyeball 12.

The vibration is transmitted to the eyeball 12 through the shaft 17 or protuberance as a known sinusoidal force applied over a range of frequencies. The amount of energy applied, in combination with a distance traveled by the protuberance 17 is related to the force response in the eyeball 12. The movement of the protuberance 17 is directly related to the movement of the eyeball 12. The movement of the eyeball 12 is measured to provide a force and phase relative to the applied phase or phase lag, to calculate the vibrational impedance.

It is contemplated that a spring biased protuberance driven by a solenoid coil would induce vibration in the eyeball 12 and permit measurement of the vibrational responses at a mechanically coupled force transducer.

Typically, the vibrator or solenoid causes a minimal displacement of the cornea, being approximately 1 μ. The range of frequencies of interest is typically from about 10 Hz to about 100 Hz.

### EXAMPLE 1

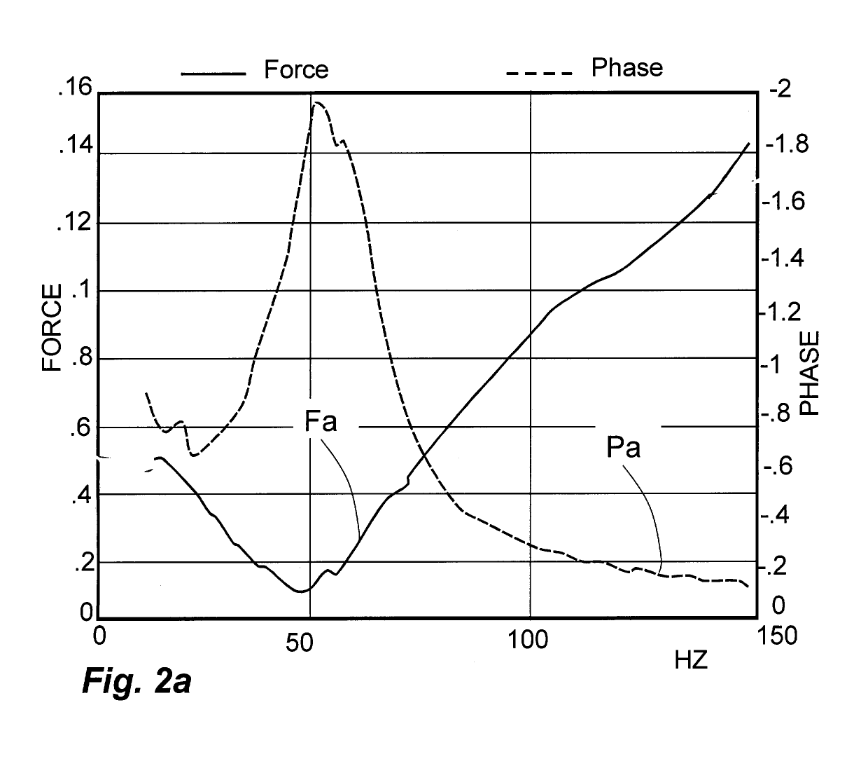

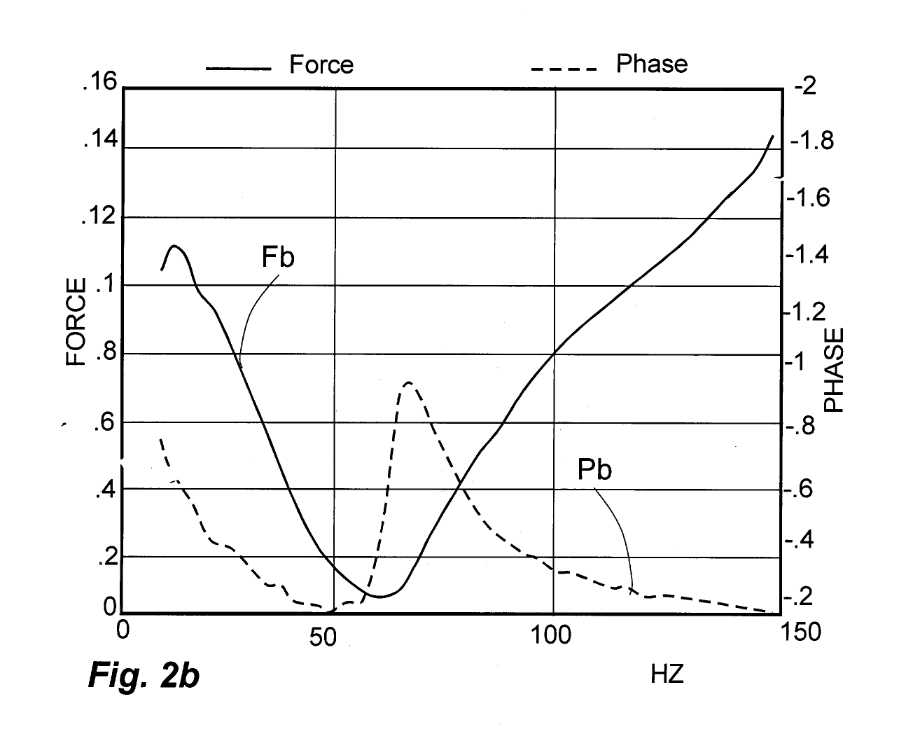

Referring to FIG. 2a, apparatus as described herein was used to measure IOP in a porcine eyeball having a low IOP. Trace Fa illustrates the amplitude of the force response in the eyeball upon applying a constant amplitude, vibrational excitation over the range of frequencies of interest. Trace Pa illustrates the corresponding phase response between the excitation oscillator and the oscillations of the force required to induce vibration in the eye.

The vibrational impedance is characterized by an inflection in the phase lag Pa which corresponds with a minimum inflection in the force trace Fa.

### EXAMPLE 2

Referring to FIG. 2b, apparatus as described herein was used to measure IOP in a porcine eyeball having a high IOP. Trace Fb illustrates the amplitude of the force response in the eyeball upon applying a constant amplitude, vibrational excitation over the range of frequencies of interest. Trace Pb illustrates the corresponding phase response between the excitation oscillator and the oscillations of the force required to induce vibration the eye.

A comparison of Examples 1 and 2 demonstrates that the eye having a higher IOP has less phase lag than an eye having lower IOP. Further, at higher IPO, there is a shift in the frequency Hz at which the inflection points of both the phase P and the force response F are manifest. In other words, the frequencies (Hz) at which the amplitude of the force F reaches a minimum and at which the phase lag P reaches a maximum, increase with increased IOP.

In a preferred use of the tonometer of the present invention, a first measurement of IOP using the vibrational impedance measurement of IOP, is compared to a known and coincident IOP measurement, such as measured using a Goldman applanation tonometer and performed at the same time by a patient's physician. A comparison between the two measurements is made for determining at least a single calibration factor which defines the relationship between the two measurements and which is specific for the individual patient. The vibrational impedance tonometer is calibrated to reflect the determined relationship and to provide repeated, accurate and calculated IOP measurements. Subsequent calibrated measurements are then performed by the patient who can notify the physician should the results fall within an unacceptable range predetermined by the physician.

Optionally and coincident with the impedance measurement, a laser interferometer may be used to gather additional properties of the eye to normalize for variations between eyes. The laser interferometer is capable of measuring the axial length of the eyeball, from which a volume of the eye is deduced. Further, a corneal thickness can be measured, from which the elasticity of the eyeball is deduced. Each eye has a different volume and mechanical properties such as elasticity, therefore these variances can be taken into consideration when calculating IOP. To do this, laser interferometry similar to that described in U.S. Pat. No. 6,288,784 to Hitzenberger et al. is used to accurately measure the corneal thickness. The entirely of U.S. Pat. No. 6,288,784 is incorporated herein by reference. Corneal thickness is related to corneal stiffness, a source of error in contact tonometry. Axial length of the eye is related to the eye's volume. Using the additional properties so measured, the eye's vibrational response is normalized with the axial length and corneal thickness to yield a more accurate IOP.

While the actual normalization of the eye's characteristics may be numerically determined, it is understood that a better measure of the IOP can be determined as a function of some basic variables including:

Ro is a function of V, Ri, and k1;

E is a function of P, H₂O, k2; and

IOP is a function of V, E, Rik3.

Where:

Ro=is the vibrational response of the eye;

V=Eye volume (axial length);

Ri=Biomechanical rigidity of the eye;

E=Elastic modulus of the eye;

P=Thickness of the cornea;

H2O=Water content of the cornea (which is substantially constant); and

k1, k2 and k3=Constants.

The determination of IOP is a multivariate analysis which is dependent upon a large body of empirical data. Practically, the resulting relationships are complex and the effects of the various parameters which affect the IOP pressure measurement have to be found empirically and preferably with the use of finite element analysis. As those of skill in the art are aware, a variety of numerical techniques can be applied to obtain the solution. One approach is to apply neural networks and statistical methods to establish these relationships and to confirm the results of finite element analysis.

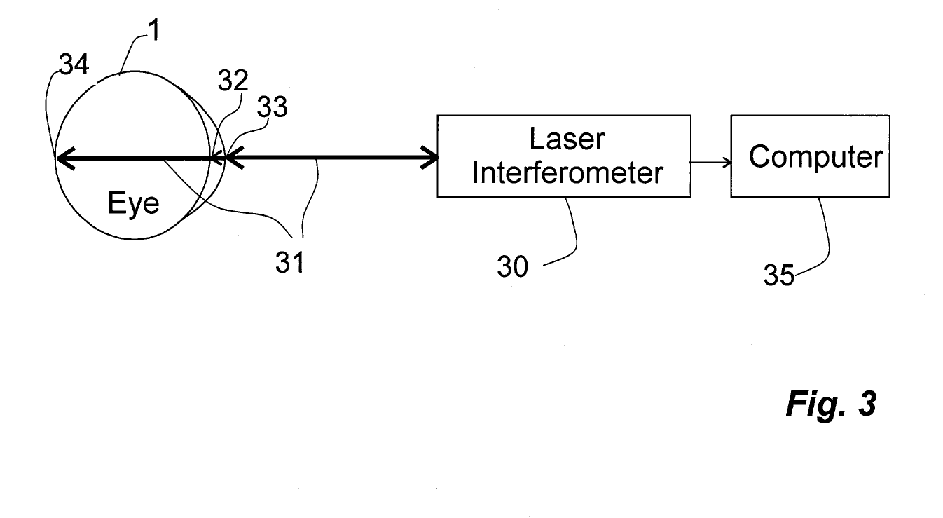

Referring to FIG. 3, additional apparatus is provided for the measurement of axial length and corneal thickness of an eyeball 12. Preferably, a laser interferometer 30 is used. A laser light beam 31 is shone into the eyeball 12 and is reflected back from inner and outer corneal surfaces 32,33 and from the back 34 of the eyeball 12 causing interference patterns. The interferometer 30 measures the interference patterns and determines path lengths to the inner and outer corneal surfaces 32,33 and to the back 34 of the eyeball 12. A computer or microprocessor 35, is used to control the interferometer 30 and to calculate the axial length and the cornea thickness.

The embodiments of the invention in which an exclusive property or privilege is claimed re defined as follows:

1. A method of determining intraocular pressure in an eyeball comprising:
contacting an eyelid with a mechanical energy transmission means capable of producing a constant amplitude and variable frequency output for inducing vibration in at least a portion of an underlying eyeball;
providing means for measuring a vibrational response in the eyeball for establishing measures indicative of vibrational impedance; and
calculating the intraocular pressure as a function of the vibrational impedance.

2. The method as described in claim 1 wherein the vibrational response is at least one of a force response and a phase lag response.

3. The method as described in claim 1 wherein the mechanical energy transmission means is a vibrator.

4. The method as described in claim 3 wherein the vibrator is a solenoid driven by an oscillator and controlled so as to provide a constant and known amplitude over a range of frequencies of vibration.

5. The method as described in claim 4 wherein the oscillator is an audio frequency oscillator controlled by a microprocessor.

6. The method as described in claim 2 wherein the means for measuring at least one of a force response and a phase response in the eyeball is a force transducer.

7. The method as described in claim 6 wherein the vibrator and the force transducer are mechanically coupled.

8. The method as described in claim 1 further comprising:
comparing a calculated intraocular pressure calculated as a function of vibrational impedance to a coincident and known intraocular pressure measurement;
establishing a relationship between the calculated intraocular pressure and the known intraocular pressure measurement for determining at least a single calibration factor; and
applying the at least a single calibration factor to subsequent vibrational impedance intraocular pressure measurements for determining the intraocular pressure.

9. A force feedback tonometer comprising:
a mechanical energy transmission means capable of producing a constant amplitude, variable frequency output for inducing vibration in at least a portion of an eyeball when positioned against an eyelid overlying the eyeball;
a device for measuring a vibrational response in the eyeball for establishing measures indicative of vibrational impedance; and
means for calculating the intraocular pressure as a function of the measures indicative of vibrational impedance.

10. The force feedback tonometer as described in claim 9 wherein the mechanical energy transmission means is a vibrator.

11. The force feedback tonometer as described in claim 10 wherein the vibrator is a solenoid driven by an oscillator and controlled so as to provide a constant and known amplitude vibration over a range of frequencies.

12. The force feedback tonometer as described in claim 11 wherein the oscillator is an audio frequency oscillator controlled by a microprocessor.

13. The force feedback tonometer as described in claim 9 wherein the vibrational response measured in the eyeball is at least one of a force response and a phase lag response.

14. The force feedback tonometer as described in claim 9 wherein the means for measuring the vibrational response in the eyeball is a force transducer.

15. The force feedback tonometer as described in claim 9 wherein the means for calculating the calculated intraocular pressure as a function of the measures indicative of vibrational impedance is a microprocessor.

16. The force feedback tonometer as described in claim 9 further comprising a static force sensor for establishing an acceptable application force of the tonometer on the eyelid.

17. The force feedback tonometer as described in claim 9 further comprising means for applying at least a single calibration factor calculated as a result of comparison of coincident measurements of intraocular pressure using vibrational impedance and a conventional intraocular pressure measurement technique to subsequent vibrational impedance measurements for determining the intraocular pressure.

18. The force feedback tonometer as described in claim 17 wherein the means for applying the at least a single calibration factor is a microprocessor.

19. The force feedback tonometer as described in claim 17 wherein the means for determining the intraocular pressure as a function of the measures indicative of vibrational impedance and the means for applying at least a single calibration is a microprocessor.
